(19) United States
(12) Patent Application Publication
(10) Pub. No.: US 2003/0170312 A1
(43) Pub. Date: Sep. 11, 2003

(54) Method for coating solid particles with a thermofusible agent, and resulting coated solid particles

(76) Inventors: Hassan Benameur, Munster (FR); Philippe Barthelemy, Mions (FR)

Correspondence Address: Sanford E Warren, Warren & Kennedy, Suite 910, 6565 MacArthur Boulevard, Irving, TX, 75039-2461, US

(21) Appl. No.: 10/333,501

(22) PCT Filed: Jul. 20, 2001
(86) PCT No.: PCT/FR01/02365

(30) Foreign Application Priority Data
Jul. 21, 2000 (FR) 00.09584

(51) Int. Cl. A61K 9/16; A61K 9/50; B05D 3/00; B01J 13/00; A61K 31/192

(52) U.S. Cl. 424/490; 427/2.15; 514/256; 514/570; 514/569

(57) ABSTRACT

The invention concerns a method for coating solid particles with a thermofusible agent which consists in: fluidizing the solid particles in an ascending air movement in spiral rotation to obtain a homogeneous individualised distribution of the particles in the air fluidized bed, the temperature of the air fluidized bed being lower than the melting point of the thermofusible agent; spraying on the particles the melted thermofusible agent in the form of atomised droplets, said droplets being distributed in a spraying cone included in an air zone, whereof the temperature enables to maintain, throughout said spraying process, a temperature of the thermofusible agent substantially equal its melting point, the spraying being carried out in the same direction and tangentially to the movement followed by the solid particles; finally, after the coating process, cooling the resulting coated particles so as to solidify the thermofusible agent around the particles.

The invention relates to a process for coating solid particles with a hot-melt agent. It also relates to the coated solid particles thus obtained.

In the remainder of the description and in the claims, the expression “hot-melt agent” refers to a raw material capable of changing, under the effect of heat, from a solid state to a liquid state, via a softening stage. The state change temperatures vary, of course, as a function of the raw material used.

Similarly, the expression “solid particles” is intended to denote single individualized particles containing a single constituent, to be distinguished therefore from the granules containing several constituents, at least one of which is a binder, intended to bind the individualized particles to one another. Of course, the particles of the invention, when they are used as a mixture, may each contain a constituent which is different in nature.

By way of raw material of this type, the use of lipid material, i.e. of a material based on free fatty acids and/or on fatty acid esters, is described hereinafter, but in a non-limiting way.

The technique termed “hot-melt coating” is a technique completely known to those skilled in the art, which consists, mainly, in spraying, while hot, fine droplets of a hot-melt coating solution onto solid particles maintained in a fluidized air bed.

The document entitled CHARACTERIZATION OF A HOT MELT FLUID BED COATING PROCESS FOR FINE GRANULES by JOZWIAKOWSKI, published in the journal Pharmaceutical Research, Volume 7, Number 11 of 1990, carries out this technique in a machine sold by GLATT under the trade name GPCG-5®. In this type of machine, the lipid coating solution is sprayed against the current of the ascending vertical air flow which maintains the particles in suspension so as to form the fluidized bed. More specifically, the spraying of the coating agent is carried out at the top of the air bed (top spray) at a high atomization air pressure, of between 4 and 5 bar, and at a coating material temperature which is from 40 to 50° C. higher than the melting point thereof (64° C.). In addition, it is indicated that the temperature of the powder bed should be maintained close to the melting temperature of the coating agent, namely, in the case in point, equal to 54° C.

It is noted, first of all, that this process requires the use of high temperatures. In addition, the fact that the droplets of coating material move against the current of the particle flow makes the coating random and difficult to control. This random coating leads to the proportion of coating material being increased in the hope of coating the particles as evenly as possible, thus increasing the cost of the process.

Accordingly, in the abovementioned document, the coating consists of lipid material representing 30% by weight of the coated particle. In addition, while such a proportion is entirely suitable for controlling the release of the active principle, it is, on the other hand, incompatible with immediate release of this active principle. Moreover, the considerable proportion of coating agent decreases all the more the concentration of active principle of the final pharmaceutical formula, causing the weight of the final form to be increased if a high content of active principle is required.

In addition, it is observed that it is difficult to maintain a constant temperature of the molten material, first of all at the spraying nozzle outlet, and then in the fluidized bed, since said temperature decreases at the time of atomizing the coating material, and then in contact with the colder air arriving against the current. One direct consequence is that a portion of the lipid material solidifies before even coming into contact with the particles, reducing the evenness of the coating and causing the formation of a solid fine powder of coating agent. These phenomena explain the choice of a temperature which is higher by 50° C. than the melting temperature of the coating agent, such that it does not have time to solidify before it contacts the particle to be coated and, on the contrary, attains a perfectly liquid state above its melting point. However, this increase in temperature which is largely above the melting temperature of the coating agent can lead to a phenomenon of aggregation and therefore of increase in particle size.

Another drawback of this technique is not being able to obtain even coating of particles of small diameter, less than 200 micrometers, without causing phenomena of aggregation of the particles among themselves (granulation).

Finally, it has been noted that this process does not make it possible to coat heat-sensitive particles, in particular those with a melting temperature close to that of the hot-melt agent, since the hot-melt agent comes into contact with the particle at a temperature considerably higher that its melting point (approximately 50° C. higher). Consequently, softening of the particle is observed which is too great to allow the coating thereof. Accordingly, to the knowledge of the applicant, all heat-sensitive particles, and in particular all active principles having a low melting point, are coated, while cold, exclusively with cellulose polymers. Document U.S. Pat. No. 4,835,187 describes, for example, a process for coating particles of ibuprofen, which has a melting point of 75° C., with an ethylcellulose solution using the technique termed “spray drying”.

In order to overcome the problems linked to the movement of the particles with respect to that of the coating agent, and the random coating which results therefrom, it has been proposed to spray the hot-melt material onto the particles not against the current of the air flow, but in the same direction as said air flow, i.e. in an ascending vertical movement (bottom spray principle, a technique of implementation of which is named WURSTER). However, the results are not satisfactory, in particular when coating fine particles is involved. Specifically, the current of air implemented causes an acceleration of the particles as a block, leading to the aggregation thereof (granulation). Moreover, this technique does not make it possible to resolve any further the problem demonstrated using top spray, which is that of the solidifying of the lipid material on contact with the current of cold air.

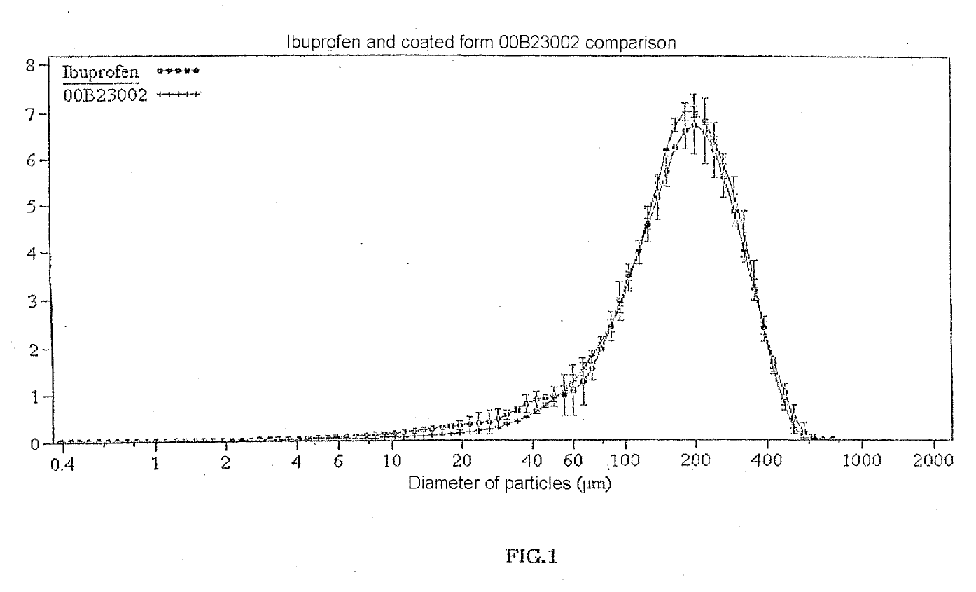

The FAHAM document, published in DIE PHARMAZIE vol. 55, Jun. 2000, pages 444-448, describes a third type of coating process, consisting not in spraying the coating solution against or in the same direction as the stream of ascending vertical air, but perpendicular to said stream of air, as shown in FIG. 1 of that document. This technique is known as “tangential spray” and gives rise to a device marketed by GLATT under the name GPCG1. This device is equipped, as shown in the figure, with a revolving disk, the stream of air circulating according to an ascending movement between the edge of the disk and the wall of the device. The high-speed rotation of the disk confers a centrifugal force on the product to be coated, the effect of which is to adhere the product to the wall of the device or to compress it against the wall of the device. Such a process therefore has the disadvantage of increasing the size of the product to be coated via a phenomenon of granulation before the coating per se. In addition, the coating as described is carried out on granules obtained by prior granulation, and not on solid individualized particles within the meaning of the invention. It is therefore impossible, using this technique, to coat particles which are small in diameter, less than 200 μm. It is observed, moreover, that the percentage of particles coated at 6%, the size of which is less than 200 μm, decreases by close to half relative to the uncoated particles (Table 2). Moreover, this process does not make it possible to coat thermosensitive particles due to the fact that, as indicated previously, the hot-melt agent comes into contact with a granule at a temperature very much higher than its melting point.

This being the case, the first problem that the invention proposes to resolve is to develop a “hot-melt coating” process which may be carried out at lower and better controlled temperatures so as to make it possible to reduce the energy consumed.

A second problem that the invention proposes to resolve is to develop a process which makes it possible to obtain a uniform and even hot-melt material coating of solid particles, using an amount of raw material which is as small as possible depending on the objective sought.

Thus, for example, when the solid particle is an active principle, the objective of the invention is to coat the particle with as little material as possible, whether for obtaining immediate or controlled release of the active principle.

A third problem that the invention proposes to resolve is to develop a coating process which may be applied to particles small in size, in practice less than 200 micrometers, without requiring prior granulation.

A fourth problem that the invention proposes to resolve is to develop a coating process which may be applied to heat-sensitive particles which have a melting point close to, but higher than, the melting point of the coating agent.

To do this, the invention provides a process for coating solid particles with at least one hot-melt agent, according to which:

the solid particles are fluidized in a spiralling, ascending current of air making it possible to obtain a homogeneous separated distribution of the particles in the air bed, the temperature of the air bed being lower than the melting temperature of the hot-melt agent,

the molten hot-melt agent is then sprayed onto the particles, in the form of atomized droplets, said droplets being distributed in a spray cone contained in a region of air, the temperature of which makes it possible to maintain, throughout said spraying, a hot-melt agent temperature which is substantially equal to the melting temperature thereof, the spraying being carried out in an ascending manner in the same direction as and tangentially to the path of the solid particles,

finally, when the coating is finished, the coated particles obtained are cooled so as to solidify the hot-melt agent around the particles.

This process can be carried out in a machine of the type of that described in document U.S. Pat. No. 5,282,321, reproducing both the movement of the particles and that of the coating agent, described above.

In other words, the invention consists in combining a first step of fluidization of the solid particles in a movement which makes it possible to obtain a completely homogeneous separation and distribution of the particles, with a second step of spraying which is tangential, and also ascending and in the same direction, under conditions such that the hot-melt agent close to the melting temperature thereof may be in immediate contact with the particles, thus decreasing any risk of rapid cooling and therefore of premature solidifying of the hot-melt agent. This process makes it possible not only to obtain uniform coating, but also to work at temperatures close to the melting temperature of the hot-melt agent.

In addition, maintaining the hot-melt agent temperature close to the melting point thereof throughout spraying makes it possible to coat heat-sensitive particles which have a melting point close to, but higher than, the melting point of the coating agent. Specifically, the coating agent comes into contact with the particle in the softened state, corresponding to the melting point thereof, and not in the liquid state, corresponding to a higher temperature, such that it is not hot enough to modify the physical state of the particle.

In addition, the specific movement of the particles within the air bed, which remain individualized with no agglomeration phenomenon associated with spraying carried out according to a similar movement, makes it possible to coat separated particles small in diameter, less than 200 micrometers, advantageously between 30 and 180 micrometers. Of course, the particle size of less than 200 micrometers is not a limiting factor for carrying out the process, it being possible to carry out this process for larger particle sizes. Moreover, it should be mentioned that the diameter indicated corresponds to the mean diameter of a population of particles.

In order to decrease the degree of softening of the particle upon contact with the molten hot-melt agent, the temperature of the air bed is advantageously chosen so as to maintain the solid particle and its environment at a temperature which is below the melting temperature of the hot-melt agent, and which advantageously has a value close to 20° C. lower than the melting temperature of the hot-melt agent. Of course, the temperature of the air may vary by a few degrees throughout the process, in particular when the hot-melt agent comes into contact with the solid particles.

In order to maintain the hot-melt agent at the melting temperature thereof throughout the spraying step, the temperature of the region of air surrounding the spray cone in which the atomized droplets are maintained is advantageously chosen between + or −5° C. with respect to the melting temperature of the hot-melt agent.

According to another characteristic of the process of the invention, the air pressure for atomizing the hot-melt agent is set, beforehand, between 0.3 bar and 5 bar, advantageously between 1 and 2 bar. Of course, those skilled in the art will adjust the atomization pressure as a function of the nature and of the Theological characteristics of the coating to be sprayed.

Moreover, the temperature of the air for atomizing the hot-melt agent is a maximum of 10° C. higher than the melting temperature of said agent.

According to another characteristic, the pressure of the region of air surrounding the cone containing the atomized droplets is preferably less than 1.5 bar, advantageously equal to 0.5 bar.

Moreover, and according to another characteristic, the spraying flow rate for the hot-melt agent is between 5 and 500 g/minute. Once again, those skilled in the art will regulate the rate as a function of the nature and of the rheological characteristics of the coating agent, and also as a function of the mass of the particles to be coated and of the size thereof.

Thus, for example, for a mass to be coated of 2,000 g, of which the size of the constituent particles is between 30 and 180 micrometers, the spraying flow rate will be advantageously chosen between 5 and 50 g/minute.

Another advantage of the invention is to decrease the proportion of the coating agent, and therefore the cost of the composition, in so far as the homogeneous distribution of the particles in the fluidized bed, combined with the control of the coating agent temperature, leads to the production of an even coating.

In practice, the coating represents from 1 to 25% by weight of the coated particle, depending on the objective sought. Thus, the coating represents between 5 and 8% when the objective is to mask the taste of an active principle, and 10 to 20% when the objective is to prolong the release of an active principle.

Of course, the process of the invention relates to any type of solid particle intended to be coated.

However, and in an advantageous embodiment, the solid particle is an active principle chosen from the group comprising: hydrochlorothiazide, acetazolamide, acetylsalicylic acid, allopurinol, alprenolol, amiloride, an anti-arrhythmia agent, an antibiotic, an antidiabetic, an anti-epileptic, anti-clotting agents, an antimycotic agent, atenolol, bendroflumethiazide, benzbromarone, benzthiazide, betamethasone and the esters thereof, a bronchodilator, buphenine, bupranolol, chlordiazepoxide, chloroquine, chlorothiazide, chlorpromazine, chlortalidone, clenbuterol, clomipramine, clonidine, co-dergocrine, cortisone, and the esters thereof, dexamethasone, and the esters thereof, dextropropoxyphene, diazepam, diazoxide, diclofenac, diclofenamide, digitalis glycoside, dihydralazine, dihidroergotamine, diltiazem, metal salts, ergotamine, ethacrynic acid, ethinyloestradiol, ethoxyzolamide, fenoterol, fludrocortisone, and the esters thereof, fluphenazine, furosemide, gallopamil, guanethidine, a hormone, hydrocortisone, and the esters thereof, hydroflumethiazide, an immunosuppressor, ibuprofen, imipramine, indomethacin, levodopa, a lithium salt, a magnesium salt, medroxyprogesterone acetate, menadione, methaqualone, 8-methoxypsoralen, methylclothiazide, methyldopa, methylprednisolone, methyltestosterone, methylthiouracil, methylxanthine, metipranolol, molsidomine, morphine, naproxen, nicergoline, nifedipine, norfenefrine, oxyphenbutazone, papaverine, parmathasone, and the esters thereof, pentobarbital, perphenazine, phenobarbital, phenylbutazone, phytomenadione, pirenzepine, polythiazide, prazosine, prednisolone, and the esters thereof, prednisone, and the esters thereof, probenecid, propranolol, propylthiouracil, rescinnamine, reserpine, secbutabarbital, secobarbital, spironolactone, sulphasalazine, sulphonamide, thioridazine, triamcinolone, and the esters thereof, triamteren, trichlormethiazide, trifluoperazine, trifluopromazine, a tubercular static agent, verapamil, a virustatic agent, a zytostatic agent, bromocriptine, bromopride, carbidopa, carbocromen, quinine, chlorprothixene, cimetidine, clofibrate, cyclizine, desipramine, disulphiram, domperidone, doxepin, fenbufen, flufenamine acid, flunarizine, gemfibrocil, haloperidol, ketoprofen, labetalol, lorazepam, mefenamine acid, melperone, metoclopramide, nortriptyline, noscapine, oxprenolol, oxymetholone, pentazocine, pethidine, stanozolol, sulindac, sulpiride, tiotixene, this list being non-limiting.

Moreover, and as already mentioned, the expression “hot-melt agent” refers to a raw material capable of changing from the solid state to the liquid state, via softening, under the effect of the temperature.

In an advantageous embodiment of the process, the hot-melt agent is a lipid, i.e. a raw material based on free fatty acids and/or on fatty acid esters, preferably comprising at least one partial ester of alcohol with at least one fatty acid.

According to a first embodiment, the lipid is an ester of behenic acid and of alcohol, sold by the applicant under the trade name COMPRITOL®. The melting temperature of COMPRITOL® ranges between 69 and 74° C. and makes it possible to coat heat-sensitive particles which have a melting point which is close but higher, for example ibuprofen, which has a melting point equal to 75° C.

In a second embodiment, the lipid agent is an ester of palmitostearic acid and of alcohol, sold under the trade name PRECIROL ATO 5® and which has a melting point ranging between 53 and 57° C.

Of course, the invention relates to the coated solid particle which can be obtained using the process described hereinabove.

A subject of the invention is also a solid particle coated with a coating agent comprising at least one partial ester of alcohol with at least one fatty acid. This particle is characterized in that the size thereof before coating is less than 400 μm, advantageously less than 200 micrometers, and in that the coating represents between 1 and 25% by weight of the coated particle.

In an advantageous embodiment, the coating represents from 2 to 8% by weight of the coated particle.

According to another characteristic, the particle is heat-sensitive and has a melting point which is close to, but higher than, that of the hot-melt agent.

According to a particular embodiment, the particle is an active principle chosen from the group of the active principles cited above.

The coating is lipid in nature and chosen preferably from the group comprising esters of palmitostearic acid and of alcohol, and esters of behenic acid and of alcohol.

The invention also relates to any composition which integrates the coated particles described hereinabove.

In a particular embodiment, a subject of the invention is an ibuprofen particle coated with a coating agent, which is characterized in that the uncoated particle size is less than 200 micrometers, and in that the coating agent comprises at least one partial ester of alcohol with at least one fatty acid and represents between 1 and 25% by weight of the coated particle, advantageously between 2 and 8%.

Of course, and as previously, the diameter of the particles defined hereinabove corresponds to a mean diameter of a given population of particles.

In an advantageous embodiment, the coating agent is chosen from the group comprising esters of palmitostearic acid and of alcohol, and esters of behenic acid and of alcohol.

Of course, the coated particles can be integrated directly into sachets or gelatin capsules, or incorporated into tablets, without this list being limiting.

The invention and the advantages which ensue therefrom will emerge more clearly from the examples of implementation hereinafter, supporting the attached figures according to which:

FIG. 1 is a representation of the distribution of a batch of coated and uncoated ibuprofen particles;

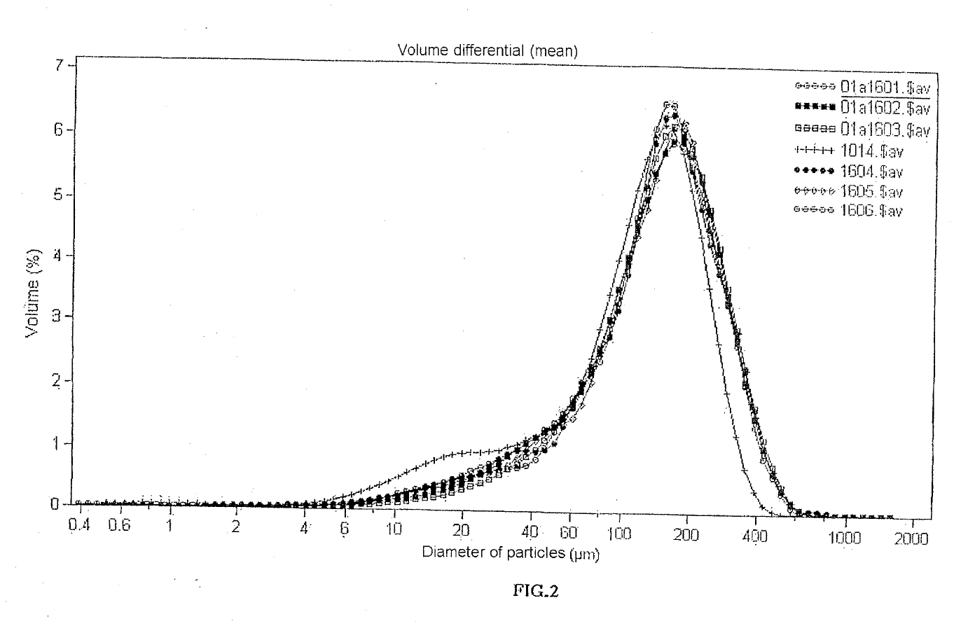

FIG. 2 is a representation of the distribution of several batches of coated and uncoated ibuprofen particles;

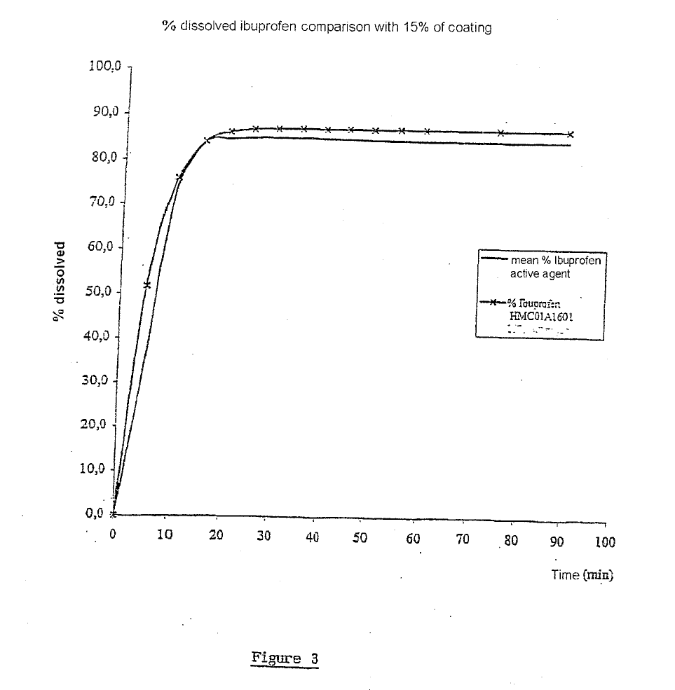

FIG. 3 is a curve of dissolution of coated and uncoated ibuprofen;

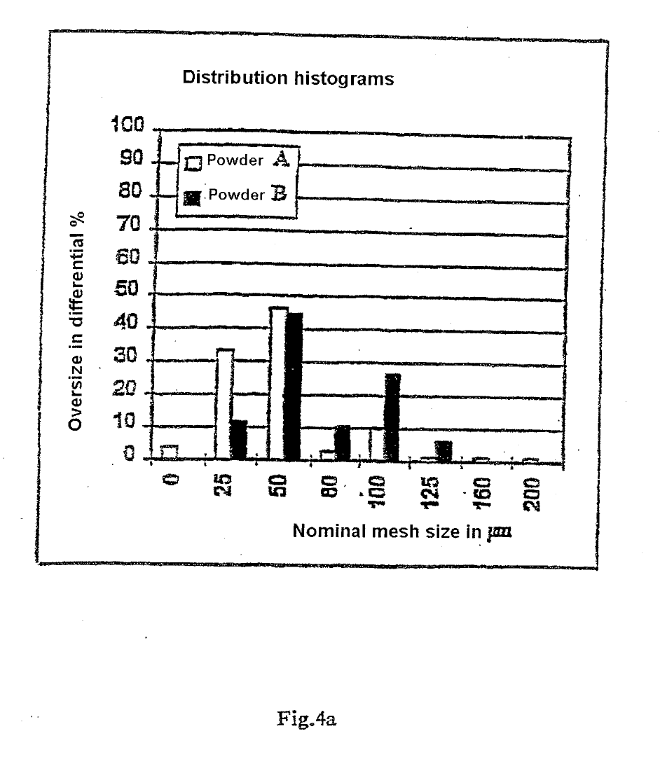

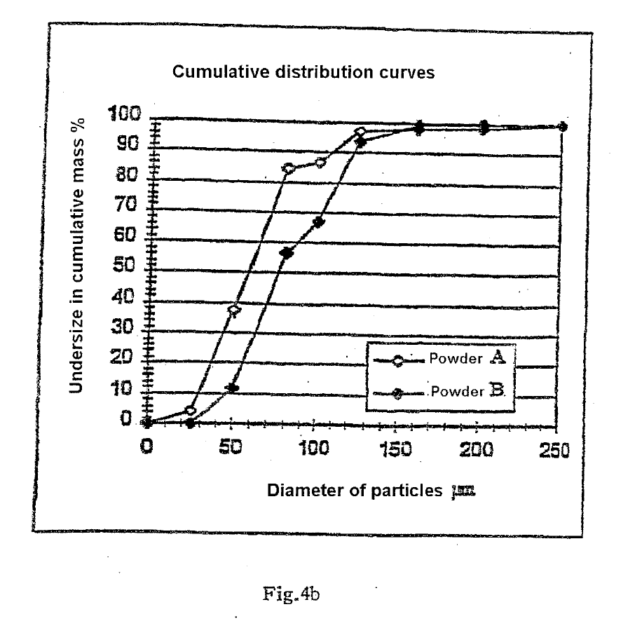

FIG. 4 is a representation of the distribution of the uncoated (powder A) and coated (powder B) ion exchange resin (IER) spherical particles, by means of a distribution histogram (4a) and of a cumulative distribution curve (4b).

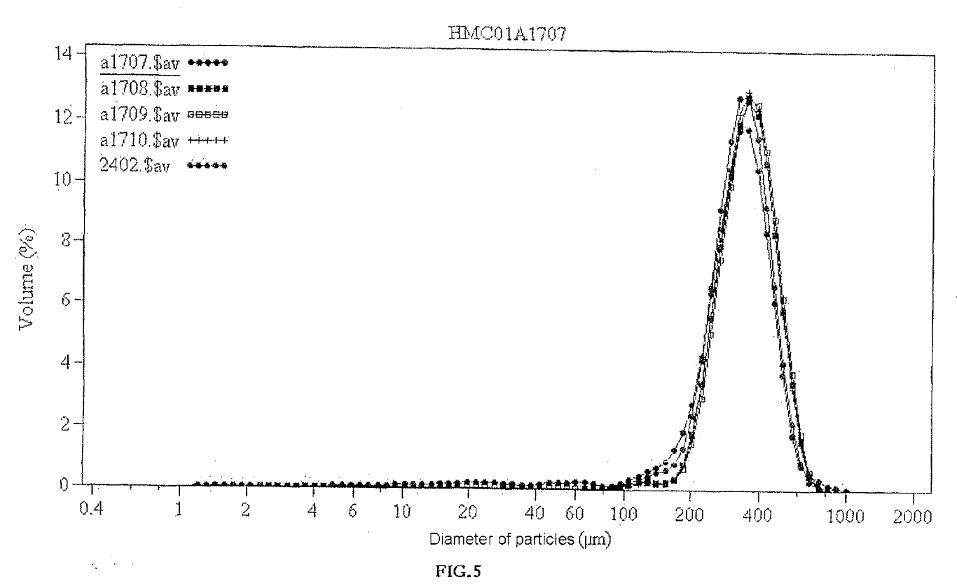

FIG. 5 is a representation of the distribution of batches of coated and uncoated paracetamol particles.

### EXAMPLE 1

Coated Ibuprofen

In this example, 2,000 g of ibuprofen, the mean diameter of the particles of which is equal to 176 micrometers, are coated with 146 g of PRECIROL ATA 5®, the coating therefore representing 7% by weight of the total weight of the coated particle. It is recalled that the melting temperature of PRECIROL ATA 5® is between 52 and 57° C., whereas the melting temperature of ibuprofen is equal to 75° C.

The process is carried out in a device named KUGELCOATER® sold by the company HUTTLIN. The KUGELCOATER® model used is the UNILAB-05.

In this example, the characteristics used throughout the process are given in the table hereinafter.

| | | | | | | Pressure of | | |
|---|---|---|---|---|---|---|---|---|
| | | Air | | Spraying | Atomization | region of air | Temp. of | Temp. of region of |
| | Air flow | bed | Particle | flow | pressure ± | surrounding | air for | air surrounding |
| Duration | rate | temp. | temp. | rate | 0.1 | the spray cone | atomization | the spray cone |
| (min) | (Nm³/h) | (° C.) | (° C.) | (g/min) | (bar) | (bar) | (° C.) | (° C.) |
| 1 | 158 | 35.0 | 36.0 | 6 | 1.0 | 0.5 | 60 | 50 |
| 4 | 161 | 34.6 | 38.4 | 6 | 1.0 | 0.5 | 60 | 50 |
| 10 | 171 | 35.0 | 39.7 | 6 | 1.0 | 0.5 | 60 | 50 |
| 15 | 168 | 35.3 | 40.1 | 6 | 1.0 | 0.5 | 60 | 50 |
| 23 | 166 | 35.3 | 40.1 | 6 | 1.0 | 0.4 | 60 | 50 |
| 32 | 142 | 22.0 | 33.2 | 6 | 0.4 | 0.4 | 60 | 50 |
| 41 | 145 | 19.7 | 30.5 | 6 | 0.4 | 0.4 | 60 | 50 |

As shown in the table, the atomization pressure is decreased from the 32nd minute so as to allow cooling.

On FIG. 1, the distribution of the particles before and after coating has been represented. As shown in this figure, the set of coated particles has the same distribution as that of the uncoated particles, showing not only that the particles were evenly coated, but also that the amount of coating is less. Thus, it is observed that the mean diameter of the particles before coating is equal to 176 micrometers, while the mean diameter of the coated particles is equal to 180 micrometers.

It is thus noted that the process makes it possible to coat, while hot, particles which have a melting point close to that of the coating agent.

### EXAMPLE 2

The aim of this characterization is to evaluate the quality of the coating obtained on six coated ibuprofen batches.

The batches studied are hereafter referenced as HMC01A1601/HMC01A1602/HMC01A1603/HMC01A1604/HMC01A1605/HMC01A1606, produced from the active principle ibuprofen EP batch 5200I1014 coated with PRECIROL® ARO 5 batch 23907 in the proportion of 15%. The process is carried out in a device identical to that used in Example 1, with the same characteristics (flow rate, pressure, etc.).

The table below shows the mean size of the ibuprofen particles (D50) before and after coating. FIG. 2 represents in parallel the distribution of the particles before and after coating.

| Sample | Mean (μm) |
|---|---|
| 5200I1014 | 130.0 |
| HMC01A1601 | 161.0 |
| HMC01A1602 | 165.2 |
| HMC01A1603 | 169.7 |
| HMC01A1604 | 164.7 |
| HMC01A1605 | 164.7 |
| HMC01A1606 | 160.6 |

As shown in this figure, the set of coated particles has the same distribution as that of the uncoated particles, showing not only that the particles were evenly coated, but also that the quality of coating is less. Specifically, the diameter of the particles before coating is 130 μm, while, after coating, it is at most 169.7 μm (batch HMC01A1603).

A test for dissolution of coated and uncoated ibuprofen (batch HMC01A1601) was also carried out in accordance with the instructions of the pharmacopoeia.

The results appear in FIG. 3. As shown in this figure, the rate of dissolution of the coated ibuprofen is virtually identical to that of the ibuprofen alone, which proves that the coating has can influence on the release of the active principle.

### EXAMPLE 3

Coated Ion Exchange Resin (IER)

In this example, 2,000 g of IER, the mean diameter of the particles of which is equal to 60 micrometers, are coated with 350 g of COMPRITOL®. The coating therefore represents 17.5% by weight of the total weight of the coated particle.

The process is carried out in a device identical to that used above.

In this example, the characteristics used throughout the process are given in the table hereinafter.

| | | | | | | Pressure of | | |
|---|---|---|---|---|---|---|---|---|
| | | Air | | Spraying | Atomization | region of air | Temp. of | Temp. of region of |
| | Air flow | bed | Particle | flow | pressure ± | surrounding | air for | air surrounding |
| Duration | rate | temp. | temp. | rate | 0.1 | the spray cone | atomization | the spray cone |
| (min) | (Nm³/h) | (° C.) | (° C.) | (g/min) | (bar) | (bar) | (° C.) | (° C.) |
| 3 | 157 | 64.8 | 56.5 | 23 | 1.6 | 1.03 | 72 | 60 |
| 20 | 190 | 45.0 | 56.8 | 11 | 1.6 | 1.05 | 72 | 60 |
| 35 | 178 | 45.1 | 55.8 | 11 | 1.58 | 1.03 | 72 | 60 |
| 48 | 167 | 43.0 | 50.9 | 11 | 1.58 | 1.03 | 72 | 60 |

On FIG. 2, the distribution of the particles before and after coating has been represented.

As shown in this figure, the set of coated particles has the same distribution as those of the uncoated particles, showing not only that the particles were evenly coated, but also that the amount of coating is less. Thus, it is observed that the mean diameter of the particles before coating is equal to 60 micrometers, while the mean diameter of the coated particles is equal to 75 micrometers.

### EXAMPLE 4

The aim of this characterization is to evaluate the quantity of the coating obtained on four pilot coated batches of paracetamol.

The batches studied are HMC01A1707/HMC01A1708/HMC01A1709/HMC01A1710, produced from the active principle Paracetamol Rhodia batch 99292402 and the coating is Precirol ATO 5 batch 23907 in a theoretical amount of 6% by mass.

The process is carried out in a device identical to that of Example 1, and under the same conditions.

The table below represents the mean size of the ibuprofen particles (D50) before and after coating. FIG. 5 represents in parallel the distribution of the particles before and after coating.

| Sample | Median (μm) |
|---|---|
| 99292402 | 326.1 |
| HMC01A1707 | 336.1 |
| HMC01A1708 | 355.2 |
| HMC01A1709 | 361.2 |
| HMC01A1710 | 354.8 |

As shown in this figure, the set of coated particles has the same distribution as those of the uncoated particles, proving not only that the particles were evenly coated, but also that the quality of coating is less. Specifically, the diameter of the particles before coating is 326.1 μm, while, after coating, it is at most 361.2 μm (batch HMC01A1709).

The invention and the advantages which ensue therefrom emerge clearly from the description. In particular, the possibility of coating heat-sensitive particles which have a melting point close to that of the coating agent, which was not possible with the existing techniques, will be noted. The technique described also makes it possible to reduce the energy required for the process. Moreover, the process makes it possible to evenly coat particles small in diameter, less than 200 micrometers, which was not possible with the described amounts of coating agent, using other techniques.

1. The process for coating solid particles with at least one hot-melt agent, according to which:
the solid particles are fluidized in a spiralling, ascending current of air making it possible to obtain a homogeneous separated distribution of the particles in the air bed, the temperature of the air bed being lower than the melting temperature of the hot-melt agent,
the molten hot-melt agent is then sprayed onto the particles, in the form of atomized droplets, said droplets being distributed in a spray cone contained in a region of air, the temperature of which makes it possible to maintain, throughout said spraying, a hot-melt agent temperature which is substantially equal to the melting temperature thereof, the spraying being carried out in an ascending manner in the same direction as and tangentially to the path of the solid particles,
finally, when the coating is finished, the coated particles obtained are cooled so as to solidify the hot-melt agent around the particles.

2. The process as claimed in claim 1, characterized in that the solid particle is heat-sensitive and has a melting point close to, but higher than, that of the hot-melt agent.

3. The process as claimed in claim 1, characterized in that the diameter of the solid particles is less than 200 micrometers, advantageously between 30 and 180 micrometers.

4. The process as claimed in claim 1, characterized in that the temperature of the air bed is chosen so as to maintain the solid particle at a temperature which is below the melting temperature of the hot-melt agent, and which advantageously has a value close to 20° C. lower than the melting temperature of the hot-melt agent.

5. The process as claimed in claim 1, characterized in that the air pressure for atomizing the hot-melt agent is set, beforehand, between 0.3 bar and 5 bar, advantageously between 1 and 2 bar.

6. The process as claimed in claim 1, characterized in that the temperature of the region of air surrounding the spray cone in which the atomized droplets are maintained is advantageously chosen between + or −5° C. with respect to the melting temperature of the hot-melt agent.

7. The process as claimed in claim 1, characterized in that the pressure of the region of air surrounding the spray cone containing the atomized droplets is less than 1.5 bar, advantageously equal to 0.5 bar.

8. The process as claimed in claim 1, characterized in that the temperature of the air for atomizing the hot-melt agent is a maximum of 10° C. higher than the melting temperature of said agent.

9. The process as claimed in claim 1, characterized in that the rate of spraying the hot-melt agent is between 5 and 50 g/minute.

10. The process as claimed in claim 1, characterized in that the coating represents from 1 to 25% by weight, depending on the objective sought.

11. The process as claimed in claim 1, characterized in that the solid particle is an active principle chosen from the group comprising:
hydrochlorothiazide, acetazolamide, acetylsalicylic acid, allopurinol, alprenolol, amiloride, an anti-arrhythmia agent, an antibiotic, an antidiabetic, an anti-epileptic, anti-clotting agents, an antimycotic agent, atenolol, bendroflumethiazide, benzbromarone, benzthiazide, betamethasone and the esters thereof, a bronchodilator, buphenine, bupranolol, chlordiazepoxide, chloroquine, chlorothiazide, chlorpromazine, chlortalidone, clenbuterol, clomipramine, clonidine, co-dergocrine, cortisone, and the esters thereof, dexamethasone, and the esters thereof, dextropropoxyphene, diazepam, diazoxide, diclofenac, diclofenamide, digitalis glycoside, dihydralazine, dihidroergotamine, diltiazem, metal salts, ergotamine, ethacrynic acid, ethinyloestradiol, ethoxyzolamide, fenoterol, fludrocortinone, and the esters thereof, fluphenazine, furosemide, gallopamil, guanethidine, a hormone, hydrocortisone, and the esters thereof, hydroflumethiazide, an immunosuppressor, ibuprofen, imipramine, indomethacin, levodopa, a lithium salt, a magnesium salt, medroxyprogesterone acetate, menadione, methaqualone, 8-methoxypsoralen, methylclothiazide, methyldopa, methylprednisolone, methylestosterone, methylthiouracil, methylxanthine, metipranodol, molsidomine, morphine, naproxen, nicergoline, nifedipine, norfenefrine, oxyphenbutazone, papaverine, parmathasone, and the esters thereof, pentobarbital, perphenazine, phenobarbital, phenylbutazone, phytomenadione, pirenzepine, polythiazide, prazosine, prednisolone, and the esters thereof, prednisone, and the esters thereof, probenecid, propranolol, propylthiouracil, rescinnamine, reserpine, secbutabarbital, secobarbital, spironolactone, sulphasalazine, sulphonamide, thioridazine, triamcinolone, and the esters thereof, triamteren, trichlormethiazide, trifluoperazine, trifluopromazine, a tubercular static agent, verapamil, a virustatic agent, a zytostatic agent, bromocriptine, bromopride, carbidopa, carbocromen, quinine, chlorprothixene, cimetidine, clofibrate, cyclizine, desipramine, disulphiram, domperidone, doxepin, fenbufen, flufenamine acid, flunarizine, gemfibrocil, haloperidol, ketoprofen, labetalol, lorazepam, mefenamine acid, melperone, metoclopramide, nortriptyline, noscapine, oxprenolol, oxymetholone, pentazocine, pethidine, stanozolol, sulindac, sulpiride, tiotixene.

12. The process as claimed in claim 1, characterized in that the hot-melt agent is a lipid based on free fatty acids and/or on fatty acid esters.

13. The process as claimed in claim 12, characterized in that the lipid comprises at least one partial ester of alcohol with at least one fatty acid.

14. The process as claimed in claim 13, characterized in that the lipid is chosen from the group comprising esters of palmitostearic acid and of alcohol, and esters of behenic acid and of alcohol.

15. A coated solid particle which can be obtained using the process which is the subject of claim 1.

16. A solid particle coated with a coating agent comprising at least one partial ester of alcohol with at least one fatty acid, characterized in that the particle size before coating is less than 400 micrometers, advantageously less than 200 micrometers, and in that the coating represents between 1 and 25% by weight of the coated particle.

17. The particle as claimed in claim 16, characterized in that the coating represents from 2 to 8% by weight of the coated particle.

18. The particle as claimed in claim 16, characterized in that it is heat-sensitive and has a melting point which is close to, but higher than, that of the hot-melt agent.

19. The particle as claimed in claim 16, characterized in that the particle is an active principle chosen from the group comprising:
hydrochlorothiazide, acetazolamide, acetylsalicylic acid, allopurinol, alprenolol, amiloride, an anti-arrhythmia agent, an antibiotic, an antidiabetic, an anti-epileptic, anti-clotting agents, an antimycotic agent, atenolol, bendroflumethiazide, benzbromarone, benzthiazide, betamethasone and the esters thereof, a bronchodilator, buphenine, bupranolol, chlordiazepoxide, chloroquine, chlorothiazide, chlorpromazine, chlortalidone, clenbuterol, clomipramine, clonidine, co-dergocrine, cortisone, and the esters thereof, dexamethasone, and the esters thereof, dextropropoxyphene, diazepam, diazoxide, diclofenac, diclofenamide, digitalis glycoside, dihydralazine, dihidroergotamine, diltiazem, metal salts, ergotamine, ethacrynic acid, ethinyloestradiol, ethoxyzolamide, fenoterol, fludrocortinone, and the esters thereof, fluphenazine, furosemide, gallopamil, guanethidine, a hormone, hydrocortisone, and the esters thereof, hydroflumethiazide, an immunosuppressor, ibuprofen, imipramine, indomethacin, levodopa, a lithium salt, a magnesium salt, medroxyprogesterone acetate, menadione, methaqualone, 8-methoxypsoralen, methylclothiazide, methyldopa, methylprednisolone, methylestosterone, methylthiouracil, methylxanthine, metipranodol, molsidomine, morphine, naproxen, nicergoline, nifedipine, norfenefrine, oxyphenbutazone, papaverine, parmathasone, and the esters thereof, pentobarbital, perphenazine, phenobarbital, phenylbutazone, phytomenadione, pirenzepine, polythiazide, prazosine, prednisolone, and the esters thereof, prednisone, and the esters thereof, probenecid, propranolol, propylthiouracil, rescinnamine, reserpine, secbutabarbital, secobarbital, spironolactone, sulphasalazine, sulphonamide, thioridazine, triamcinolone, and the esters thereof, triamteren, trichlormethiazide, trifluoperazine, trifluopromazine, a tubercular static agent, verapamil, a virustatic agent, a zytostatic agent, bromocriptine, bromopride, carbidopa, carbocromen, quinine, chlorprothixene, cimetidine, clofibrate, cyclizine, desipramine, disulphiram, domperidone, doxepin, fenbufen, flufenamine acid, flunarizine, gemfibrocil, haloperidol, ketoprofen, labetalol, lorazepam, mefenamine acid, melperone, metoclopramide, nortriptyline, noscapine, oxprenolol, oxymetholone, pentazocine, pethidine, stanozolol, sulindac, sulpiride, tiotixene.

20. The particle as claimed in claim 16, characterized in that the partial ester of alcohol with at least one fatty acid is chosen from the group comprising esters of palmitostearic acid and of alcohol, and esters of behenic acid and of alcohol.

21. A composition which integrates the coated particles which are the subjects of claim 16.

22. An ibuprofen particle coated with a coating agent, characterized in that the uncoated particle size is less than 200 micrometers, and in that the coating agent comprises at least one partial ester of alcohol with at least one fatty acid and represents between 1 and 25% by weight of the coated particle, advantageously between 2 and 8%.

23. The particle as claimed in claim 22, characterized in that the coating agent is chosen from the group comprising esters of palmitostearic acid and of alcohol, and esters of behenic acid and of alcohol.
